(19) United States
(12) Patent Application Publication
(10) Pub. No.: US 2006/0084614 A1
(43) Pub. Date: Apr. 20, 2006

(54) Novel factor viia inhibiting compounds

(76) Inventors: Robert Eckl, Munchen (DE); Lutz Weber, Germering (DE); Christian Oefner, Freiburg (DE)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/541,347

(22) PCT Filed: Jan. 5, 2004
(86) PCT No.: PCT/EP04/00026

§ 371 Date: Jul. 5, 2005

(30) Foreign Application Priority Data
Jan. 3, 2003 (DE) 10300049.6

(51) Int. Cl. A61K 31/704; C07H 17/00

(52) U.S. Cl. 514/25; 536/17.1

(57) ABSTRACT

The present invention relates to new compounds of formula (I): 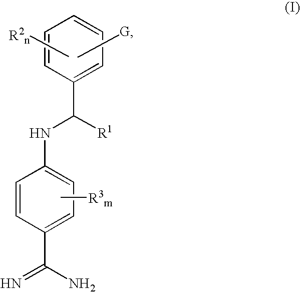 These compounds are very effective factor VIIa inhibitors and are therefore of interest in the treatment and/or prevention of thromboses, stroke, heart attack, inflammation, arteriosclerosis and tumour conditions.

The present invention relates to new compounds having an inhibitory action on blood clotting (so-called anticoagulants). These compounds are very effective factor VIIa inhibitors and are therefore of interest in the treatment and/or prevention of thromboses, stroke, heart attack, inflammation, arteriosclerosis and tumour conditions.

Thromboembolic conditions are caused by an increased tendency to blood clotting in people with risk factors, such as, for example, relatively major operations, prolonged immobilisation, fractures of the lower extremities, obesity, blood fat metabolism disorders, infections with gram-negative organisms, cancer and older age.

Venous thromboses may lead to the development of oedema or inflammation of the tissue drained by the affected vein. Thrombosis of a deeper vein (so-called deep vein thrombosis) may lead to serious complications, such as, for example, pulmonary embolism. Arterial thrombosis may lead to ischaemic necrosis of the tissue supplied by the affected artery, such as, for example, to myocardial infarct in the case of an affected coronary artery. Other thromboembolic conditions are, for example, arteriosclerosis, apoplexy (stroke), angina pectoris, intermittent claudication.

Factor VIIa inhibitors inhibit the formation, brought about by factor VIIa and tissue factor, of the clotting factors Xa, IXa and thrombin. As a result, they influence both platelet aggregation, which is brought about by those factors, and also plasma clotting. They accordingly prevent the formation of thrombi and can be used in combatting and/or preventing conditions such as thrombosis, stroke, heart attack, inflammation and arteriosclerosis. These compounds furthermore have an effect on tumour cells and prevent metastases. Consequently they can also be used as anti-tumour agents.

An object of the present invention was to provide new factor VIIa inhibitors having improved efficacy, reduced side-effects and/or increased selectivity. In addition, suitable pharmaceutical compositions were to be provided. Those compounds and compositions were to be administrable preferably parenterally or orally, especially orally.

The present invention relates to a compound of the general formula (I):

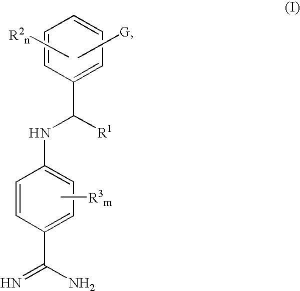

wherein

R¹ is a hydrogen atom, a heteroalkyl, heteroalkylcycloalkyl or heteroaralkyl radical,

the radicals R², each independently of any other(s), are halogen atoms, hydroxy, amino, nitro or thiol groups, alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl radicals,

the radicals R³, each independently of any other(s), are halogen atoms, hydroxy, amino, nitro or thiol groups, alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl radicals,

G is a glycosyl group,

n is 0, 1, 2, 3 or 4 and

m is 0, 1, 2, 3 or 4,

or a pharmacologically acceptable salt, solvate, hydrate or pharmacologically acceptable formulation thereof.

The expression alkyl refers to a saturated, straight-chain or branched hydrocarbon group having from 1 to 12 carbon atoms, preferably from 1 to 6 carbon atoms, especially from 1 to 4 carbon atoms, for example a methyl, ethyl, propyl, isopropyl, isobutyl, tert-butyl, n-hexyl, 2,2-dimethylbutyl or n-octyl group.

The expressions alkenyl and alkynyl refer to at least partially unsaturated, straight-chain or branched hydrocarbon groups having from 2 to 12 carbon atoms, preferably from 2 to 6 carbon atoms, especially from 2 to 4 carbon atoms, for example an ethenyl, allyl, ethynyl, propargyl, isoprenyl or hex-2-enyl group. Alkenyl groups preferably have one or two (especially one) double bond(s) and alkynyl groups preferably have one or two (especially one) triple bond(s).

Furthermore, the terms alkyl, alkenyl and alkynyl refer to optionally substituted groups in which e.g. one, two or more hydrogen atoms have been replaced by a halogen atom (preferably F or Cl) such as, for example, a 2,2,2-trichloroethyl or trifluoromethyl group.

The expression heteroalkyl refers to an alkyl, alkenyl or alkynyl group in which one or more (preferably 1, 2 or 3) carbon atoms have been replaced by an oxygen, nitrogen, phosphorus, boron, selenium, silicon or sulphur atom (preferably oxygen, sulphur or nitrogen). The expression heteroalkyl furthermore refers to a carboxylic acid or to a group derived from a carboxylic acid such as, for example, acyl, acylalkyl, alkoxycarbonyl, acyloxy, acyloxyalkyl, carboxyalkylamide or alkoxycarbonyloxy.

Examples of heteroalkyl groups are groups of formulae R^{a}—O—Y—, R^{a}—S—Y—, R^{a}N(R^{b})—Y—, R^{a}—CO—Y—, R^{a}—O—CO—Y—, R^{a}—CO—O—Y—, R^{a}—CO—N(R^{b})—Y—, R^{a}—N(R^{b})—CO—Y—, R^{a}—O—CO—N(R^{b})—Y—, R^{a}—N(R^{b})—CO—O—Y—, R^{a}—N(R^{b})—CO—N(R^{c})—Y—, R^{a}—O—CO—O—Y—, R^{a}—N(R^{b})—C(═NR^{d})—N(R^{c})—Y—, R^{a}—CS—Y—, R^{a}—O—CS—Y—, R^{a}—CS—O—Y—, R^{a}—CS—N(R^{b})—Y—, R^{a}—N(R^{b})—CS—Y—, R^{a}—O—CS—N(R^{b})—Y—, R^{a}—N(R^{b})—CS—O—Y—, R^{a}—N(R^{b})—CS—N(R^{c})—Y—, R^{a}—O—CS—O—Y—, R^{a}—S—CO—Y—, R^{a}—CO—S—Y—, R^{a}—S—CO—N(R^{b})—Y—, R^{a}—N(R^{b})—CO—S—Y—, R^{a}—S—CO—O—Y—, R^{a}—O—CO—S—Y—, R^{a}—S—CO—S—Y—, R^{a}—S—CS—Y—, R^{a}—CS—S—Y—, R^{a}—S—CS—N(R^{b})—Y—, R^{a}—N(R^{b})—CS—S—Y—, R^{a}—S—CS—O—Y—, R^{a}—O—CS—S—Y—, R^{a} being a hydrogen atom, a C₁-C₆alkyl, C₁-C₆alkenyl or C₁-C₆alkynyl group; R^{b} being a hydrogen atom, a C₁-C₆alkyl, C₁-C₆alkenyl or C₁-C₆alkynyl group; R^{c} being a hydrogen atom, a C₁-C₆alkyl, C₁-C₆alkenyl or C₁-C₆alkynyl group; R^{d} being a hydrogen atom, a C₁-C₆alkyl, C₁-C₆alkenyl or C₁-C₆alkynyl group and Y being a direct bond, a C₁-C₆alkylene, C₁-C₆alkenylene or C₁-C₆alkynylene group, each heteroalkyl group containing at least one carbon atom and it being possible for one or more hydrogen atoms to have been replaced by fluorine or chlorine atoms. Specific examples of heteroalkyl groups are methoxy, trifluoromethoxy, ethoxy, n-propyloxy, isopropyloxy, tertbutyloxy, methoxymethyl, ethoxymethyl, methoxyethyl, methylamino, ethylamino, dimethylamino, diethylamino, isopropylethylamino, methylaminomethyl, ethylaminomethyl, diisopropylaminoethyl, enol ether, dimethylaminomethyl, dimethylaminoethyl, acetyl, propionyl, butyryloxy, acetyloxy, methoxycarbonyl, ethoxycarbonyl, N-ethyl-N-methylcarbamoyl and N-methylcarbamoyl. Further examples of heteroalkyl groups are nitrile, isonitrile, cyanate, thiocyanate, isocyanate, isothiocyanate and alkylnitrile groups.

The expression cycloalkyl refers to a saturated or partially unsaturated (for example, cycloalkenyl) cyclic group having one or more rings (preferably 1 or 2) containing from 3 to 14 ring carbon atoms, preferably from 3 to 10 (especially 3, 4, 5, 6 or 7) ring carbon atoms. The expression cycloalkyl refers furthermore to groups in which one or more hydrogen atoms have been replaced by fluorine, chlorine, bromine or iodine atoms or by OH, ═O, SH, ═S, NH₂, ═NH or NO₂ groups, that is to say, for example, cyclic ketones such as, for example, cyclohexanone, 2-cyclohexenone or cyclopentanone. Further specific examples of cycloalkyl groups are a cyclopropyl, cyclobutyl, cyclopentyl, spiro[4,5]decanyl, norbornyl, cyclohexyl, cyclopentenyl, cyclohexadienyl, decalinyl, cubanyl, bicyclo[4.3.0]nonyl, 1,2,3,4-tetrahydronaphthyl, cyclopentylcyclohexyl, fluorocyclohexyl or cyclohex-2-enyl group.

The expression heterocycloalkyl refers to a cycloalkyl group as defined above in which one or more (preferably 1, 2 or 3) ring carbon atoms have been replaced by an oxygen, nitrogen, silicon, selenium, phosphorus or sulphur atom (preferably oxygen, sulphur or nitrogen). A heterocycloalkyl group has preferably 1 or 2 ring(s) containing from 3 to 10 (especially 3, 4, 5, 6 or 7) ring atoms. The expression heterocycloalkyl refers furthermore to groups in which one or more hydrogen atoms have been replaced by fluorine, chlorine, bromine or iodine atoms or by OH, ═O, SH, ═S, NH₂, ═NH or NO₂ groups. Examples are a piperidyl, morpholinyl, urotropinyl, pyrrolidinyl, tetrahydrothiophenyl, tetrahydropyranyl, tetrahydrofuryl, oxacyclopropyl, azacyclopropyl or 2-pyrazolinyl group and also lactams, lactones, cyclic imides and cyclic anhydrides.

The expression alkylcycloalkyl refers to groups containing both cycloalkyl and also alkyl, alkenyl or alkynyl groups in accordance with the above definitions, for example alkylcycloalkyl, alkylcycloalkenyl, alkenylcycloalkyl and alkynylcycloalkyl groups. An alkylcyloalkyl group preferably contains a cycloalkyl group having one or two ring(s) containing from 3 to 10 (especially 3, 4, 5, 6 or 7) ring carbon atoms, and one or two alkyl, alkenyl or alkynyl groups containing 1 or 2 to 6 carbon atoms.

The expression heteroalkylcycloalkyl refers to alkylcycloalkyl groups as defined above in which one or more (preferably 1, 2 or 3) carbon atoms have been replaced by an oxygen, nitrogen, silicon, selenium, phosphorus or sulphur atom (preferably oxygen, sulphur or nitrogen). A heteroalkylcycloalkyl group contains preferably 1 or 2 ring(s) containing from 3 to 10 (especially 3, 4, 5, 6 or 7) ring atoms and one or two alkyl, alkenyl, alkynyl or heteroalkyl groups containing 1 or 2 to 6 carbon atoms. Examples of such groups are alkylheterocycloalkyl, alkylheterocycloalkenyl, alkenylheterocycloalkyl, alkynylheterocycloalkyl, heteroalkylcycloalkyl, heteroalkylheterocycloalkyl and heteroalkylheterocycloalkenyl, it being possible for the cyclic groups to be saturated or mono-, di- or poly-unsaturated.

The expression aryl or Ar refers to an aromatic group which has one or more rings containing from 6 to 14 ring carbon atoms, preferably from 6 to 10 (especially 6) ring carbon atoms. The expression aryl (or Ar) refers furthermore to groups in which one or more hydrogen atoms have been replaced by fluorine, chlorine, bromine or iodine atoms or by OH, SH, NH₂ or NO₂ groups. Examples are a phenyl, naphthyl, biphenyl, 2-fluorophenyl, anilinyl, 3-nitrophenyl or 4-hydroxyphenyl group.

The expression heteroaryl refers to an aromatic group which has one or more rings containing from 5 to 14 ring atoms, preferably from 5 to 10 (especially 5 or 6) ring atoms, and containing one or more (preferably 1, 2, 3 or 4) oxygen, nitrogen, phosphorus or sulphur ring atoms (preferably O, S or N). The expression heteroaryl refers furthermore to groups in which one or more hydrogen atoms have been replaced by fluorine, chlorine, bromine or iodine atoms or by OH, SH, NH₂ or NO₂ groups. Examples are 4-pyridyl, 2-imidazolyl, 3-phenylpyrrolyl, thiazolyl, oxazolyl, triazolyl, tetrazolyl, isoxazolyl, indazolyl, indolyl, benzimidazolyl, pyridazinyl, quinolyl, purinyl, carbazolyl, acridinyl, pyrimidyl, 2,3′-bifuryl, 3-pyrazolyl and isoquinolyl groups.

The expression aralkyl refers to groups containing both aryl and also alkyl, alkenyl, alkynyl and/or cycloalkyl groups in accordance with the above definitions such as, for example, arylalkyl, arylalkenyl, arylalkynyl, arylcycloalkyl, arylcycloalkenyl, alkylarylcycloalkyl and alkylarylcycloalkenyl groups. Specific examples of aralkyls are toluene, xylene, mesitylene, styrene, benzyl chloride, o-fluorotoluene, 1H-indene, 1,2,3,4-tetrahydronaphthyl, dihydronaphthalene, indanone, phenylcyclopentyl, cumene, cyclohexylphenyl, fluorene and indan. An aralkyl group preferably comprises an aromatic ring system (1 or 2 rings) containing from 6 to 10 ring carbon atoms and one or two alkyl, alkenyl and/or alkynyl groups each containing 1 or 2 to 6 carbon atoms and/or a cycloalkyl group containing 5 or 6 ring carbon atoms.

The expression heteroaralkyl refers to an aralkyl group as defined above in which one or more (preferably 1, 2, 3 or 4) carbon atoms have been replaced by an oxygen, nitrogen, silicon, selenium, phosphorus, boron or sulphur atom (preferably oxygen, sulphur or nitrogen), that is to say to groups containing both aryl or heteroaryl and also alkyl, alkenyl or alkynyl and/or heteroalkyl and/or cycloalkyl and/or heterocycloalkyl groups in accordance with the above definitions. A heteroaralkyl group preferably contains an aromatic ring system (1 or 2 rings) containing 5 or 6 to 10 ring carbon atoms and one or two alkyl, alkenyl and/or alkynyl groups containing 1 or 2 to 6 carbon atoms and/or a cycloalkyl group containing 5 or 6 ring carbon atoms, 1, 2, 3 or 4 of those carbon atoms having been replaced by oxygen, sulphur or nitrogen atoms.

Examples are arylheteroalkyl, arylheterocycloalkyl, arylheterocycloalkenyl, arylalkylheterocycloalkyl, arylalkenylheterocycloalkyl, arylalkynylheterocycloalkyl, arylalkylheterocycloalkenyl, heteroarylalkyl, heteroarylalkenyl, heteroarylalkynyl, heteroarylheteroalkyl, heteroarylcycloalkyl, heteroarylcycloalkenyl, heteroarylheterocycloalkyl, heteroarylheterocycloalkenyl, heteroarylalkylcycloalkyl, heteroarylalkylheterocycloalkenyl and heteroarylheteroalkylheterocycloalkyl groups, the cyclic groups being saturated or mono-, di- or tri-unsaturated. Specific examples are a tetrahydroisoquinolyl, benzoyl, 2- or 3-ethyl-indolyl, 4-methylpyridino, 2-, 3- or 4-methoxyphenyl, 4-ethoxyphenyl, 2-, 3- or 4-carboxyphenylalkyl group.

The expressions cycloalkyl, heterocycloalkyl, alkylcycloalkyl, heteroalkylcycloalkyl, aryl, heteroaryl, aralkyl and heteroaralkyl also refer to optionally substituted groups in which e.g. one or more hydrogen atoms of such groups have been replaced by fluorine, chlorine, bromine or iodine atoms or by OH, ═O, SH, ═S, NH₂, ═NH or NO₂ groups.

The expression “optionally substituted” also refers to groups in which one or more hydrogen atoms have been replaced by fluorine, chlorine, bromine or iodine atoms or by OH, ═O, SH, ═S, NH₂, ═NH or NO₂ groups. The expression refers furthermore to groups which are substituted by unsubstituted C₁-C₆alkyl, C₂-C₆alkenyl, C₂-C₆alkynyl, C₁-C₆heteroalkyl, C₃-C₁₀cycloalkyl, C₂-C₉heterocycloalkyl, C₆-C₁₀aryl, C₁-C₉heteroaryl, C₇-C₁₂aralkyl or C₂-C₁₁heteroaralkyl groups.

In the context of the present invention, the expression glycosyl group refers to a saccharide (mono- or oligo-saccharide) bonded by way of an α- or β-O—, —S—, —N— or —C-glycosidic bond (preferably an O-glycosidic bond), preferably β-D-glucose.

Owing to their substitution, compounds of formula (I) may contain one or more centres of chirality. The present invention therefore includes both all pure enantiomers and all pure diastereoisomers and also mixtures thereof in any mixing ratio. The present invention moreover also includes all cis/trans-isomers of the compounds of the general formula (I) and also mixtures thereof. The present invention moreover includes all tautomeric forms of the compounds of formula (I).

Preference is given to compounds of formula (I) wherein R¹ is a hydrogen atom or a group of formula COOR⁴ or CONR⁵R⁶ wherein R⁴, R⁵ and R⁶ are, each independently of the others, hydrogen atoms, alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl radicals, or R⁵ and R⁶ together are part of an optionally substituted heteroaryl or heterocycloalkyl ring.

Preference is furthermore given to R⁴ being a hydrogen atom or a C₁-C₄alky or benzyl radical.

Special preference is given to R¹ being a hydrogen atom or a group of formula COOH or COOEt.

Preference is moreover given to R¹ being a group of formula CONHR⁵ wherein R⁵ is itself preferably an aralkyl (especially benzyl) or heteroaralkyl group.

Preference is furthermore given to compounds of formula (I) wherein m is 0.

Preference is also given to m being 1, R³ being especially a hydroxy group which is bonded to the phenyl ring in a position ortho to the amidino group.

Preference is moreover given to compounds of formula (I) wherein n is 2.

Preference is also given to the radicals R² being, each independently of any other(s), C₁-C₄alkyloxy, C₁-C₄hydroxyalkyloxy or benzyloxy groups; R² being especially a methoxy or ethyloxy group.

Special preference is given to compounds of the general formula (II):

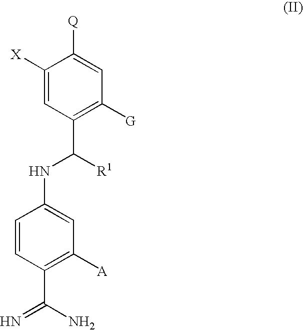

wherein X is a hydrogen atom, a C₁-C₄alkyloxy or benzyloxy group (especially a methoxy or ethoxy group); Q is a hydrogen atom, a C₁-C₄alkyloxy or benzyloxy group (especially a methoxy or ethoxy group); G is a glycosyl group (especially a β-D-glucosyloxy group); A is a hydrogen atom or a hydroxy group and R¹ is a hydrogen atom or a group of formula COOH or COOEt, or pharmacologically acceptable salts, solvates hydrates or pharmacologically acceptable formulations thereof.

Special preference is given to compounds of formulae (I) and (II) wherein the stereochemistry at the carbon atom carrying R¹ is (R) according to the Cahn-Ingold-Prelog nomenclature.

Examples of pharmacologically acceptable salts of compounds of formulas (I) or (II) are salts of physiologically acceptable mineral acids, such as hydrochloric acid, sulphuric acid and phosphoric acid; or salts of organic acids, such as methanesulphonic acid, p-toluenesulphonic acid, lactic acid, acetic acid, trifluoroacetic acid, citric acid, succinic acid, fumaric acid, maleic acid and salicylic acid. Compounds of formulas (I) or (II) can be solvated, especially hydrated. The hydration may take place, for example, during the preparation process or as a consequence of the hygroscopic nature of the initially anhydrous compounds of formulas (I) or (II).

The pharmaceutical compositions according to the present invention comprise at least one compound of formulas (I) or (II) as active ingredient and optionally carrier substances and/or adjuvants.

The pro-drugs to which the present invention also relates consist of a compound of formulas (I) or (II) and at least one pharmacologically acceptable protecting group that is removed under physiological conditions, for example an alkoxy, aralkyloxy, acyl or acyloxy group, such as, for example, a hydroxy, methoxy, ethoxy, benzyloxy, acetyl or acetyloxy group.

A compound or pharmaceutical composition of the present invention can be used in inhibiting factor VIIa activity, in the prevention and/or treatment of thromboembolic conditions, arterial restenosis, septicaemia, cancer, acute inflammation or other conditions mediated by factor VIIa activity, and especially venous thromboses, oedema or inflammation, deep vein thrombosis, pulmonary embolisms, thromboembolic complications after relatively major operations, in the case of vascular surgery, prolonged immobilisation, fractures of the lower extremities etc., arterial thromboses, especially of the coronary vessels in the event of myocardial infarct, and arteriosclerosis, stroke, angina pectoris, intermittent claudication, to mention but a few indications.

As mentioned above, the therapeutic use of the compounds of formulas (I) or (II), of their pharmacologically acceptable salts and solvates and hydrates and also formulations and pharmaceutical compositions lies within the scope of the present invention.

The present invention relates also to the use of those active ingredients in the preparation of medicaments for the prevention and/or treatment of the described conditions. In general, compounds of formulas (I) or (II) are administered either individually or in combination with any other desired therapeutic agent, using the known and acceptable methods. Such therapeutically useful compositions may be administered by one of the following routes: orally, for example in the form of dragées, coated tablets, pills, semi-solid substances, soft or hard capsules, solutions, emulsions or suspensions; parenterally, for example in the form of an injectable solution; rectally in the form of suppositories; by inhalation, for example in the form of a powder formulation or spray, transdermally or intranasally. For the preparation of such tablets, pills, semi-solid substances, coated tablets, dragées and hard gelatin capsules, the therapeutically usable product can be mixed with pharmacologically inert, inorganic or organic pharmaceutical carrier substances, for example with lactose, sucrose, glucose, gelatin, malt, silica gel, starch or derivatives thereof, talcum, stearic acid or salts thereof, skimmed milk powder and the like. For the preparation of soft capsules, pharmaceutical carrier substances such as, for example, vegetable oils, petroleum, animal or synthetic oils, wax, fat and polyols can be used. For the preparation of liquid solutions and syrups, pharmaceutical carrier substances such as, for example, water, alcohols, aqueous saline solution, aqueous dextrose, polyols, glycerol, vegetable oils, petroleum and animal or synthetic oils can be used. For suppositories, pharmaceutical carrier substances such as, for example, vegetable oils, petroleum, animal or synthetic oils, wax, fat and polyols can be used. For aerosol formulations, compressed gases that are suitable for the purpose can be used, such as, for example, oxygen, nitrogen and carbon dioxide. The pharmaceutically acceptable agents may also comprise additives for preserving and stabilising, emulsifiers, sweeteners, flavourings, salts for altering the osmotic pressure, buffers, encapsulation additives and anti-oxidants.

Combinations with other therapeutic agents may comprise other active ingredients that are customarily used for the prevention and/or treatment of thromboembolic conditions, such as, for example, warfarin etc.

For the prevention and/or treatment of the conditions mentioned above, the dose of the biologically active compound according to the invention can vary within wide limits and can be adjusted to individual requirements. In general, a dose of from 0.1 μg to 20 mg/kg of body weight per day is suitable, a preferred dose being from 0.5 to 4 mg/kg per day. In suitable cases, the dose may also be below or above the stated values.

The daily dose can be administered in, for example, 1, 2, 3 or 4 individual doses. It is also possible to administer the dose as a single dose for one week.

The compounds of the general formulas (I) or (II) described herein are distinguished with respect to the compounds described in the prior art (EP 0 921 116, WO00/35858 WO01/90051) by lower toxicity, improved activity, improved transport behaviour and better bioavailability (especially oral bioavailability).

Compounds of formulae (I) and (II) can be prepared analogously to the processes described in EP 0 921 116, WO00/35858 and WO01/90051, using suitable starting materials. Glycosylated benzaldehydes can be prepared, for example, according to the processes described in Kleine et al., Carbohydrate Research 1985, 142, 333-337 and Brewster et al., Tetrahedron Letters 1997, 5051-5054.

### EXAMPLES

The glycosylated benzaldehydes were prepared according to the procedure described in Kleine et al. Carbohydrate Research 1985, 142, 333-337. These were then reacted according to the following general working procedure (WO03064440, WO03064378): 1 mmol amine and 1 mmol aldehyde are stirred at room temperature in 20 ml acetonitrile/water (mixing ratio from 1:0 to 1:1). Subsequently, 1 mmol isonitrile is added and stirred for another 15 h. The solvent is removed in vacuo, the acetyl groups are cleaved with 2 M NH₃ in methanol and the residue is purified via HPLC. The identification of the compounds was carried out via MS.

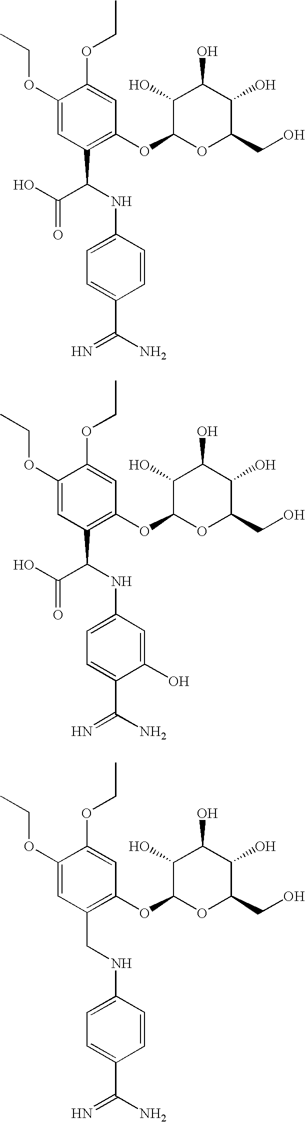

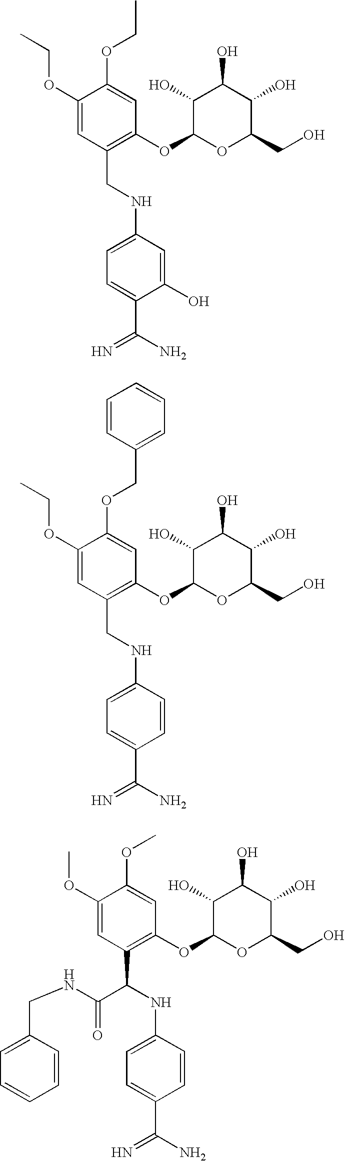

1. Compounds of the general formula (I): 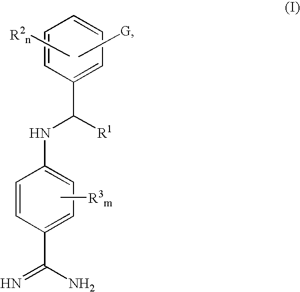
wherein
R¹ is a hydrogen atom, a heteroalkyl, heteroalkylcycloalkyl or heteroaralkyl radical,
the radicals R², each independently of any other(s), are halogen atoms, hydroxy, amino, nitro or thiol groups, alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl radicals,
the radicals R³, each independently of any other(s), are halogen atoms, hydroxy, amino, nitro or thiol groups, alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl radicals,
G is a glycosyl group,
n is 0, 1, 2, 3 or 4 and
m is 0, 1, 2, 3 or 4,
or a pharmacologically acceptable salt, solvate, hydrate or pharmacologically acceptable formulation thereof.

2. Compounds according to claim 1, wherein R¹ is a hydrogen atom or a group of formula COOR⁴ or CONR⁵R⁶ wherein R⁴, R⁵ and R⁶ are, each independently of the others, hydrogen atoms, alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl radicals, or R⁵ and R⁶ together are part of an optionally substituted heteroaryl or heterocycloalkyl ring.

3. Compounds according to claim 1, wherein R⁴ is a hydrogen atom, a C₁-C₄alkyl or benzyl radical.

4. Compounds according to claim 1, wherein R¹ is a hydrogen atom or a group of formula COOH or COOEt.

5. Compounds according to claim 1, wherein R¹ is a group of formula CONHR⁵ and wherein R⁴, R⁵ and R⁶ are, each independently of the others, hydrogen atoms, alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl radicals, or R⁵ and R⁶ together are part of an optionally substituted heteroaryl or heterocycloalkyl ring.

6. Compounds according claim 1, wherein m is 0.

7. Compounds according to claim 1, wherein m is 1 and R³ is a hydroxy group which is bonded to the phenyl ring in a position ortho to the amidino group.

8. Compounds according to claim 1, wherein n is 2.

9. Compounds according to claim 1, wherein the radicals R², each independently of any other(s), are C₁-C₄alkyloxy, C₁-C₄hydroxyalkyloxy or benzyloxy groups.

10. Pharmaceutical compositions comprising a compound according to claim 1 as active ingredient and, optionally, carrier substances and/or adjuvants.

11. A method for inhibiting blood clotting in a patient in need thereof comprising administration of an effective amount of a pharmaceutical composition as claimed in claim 10.

12. A method as claimed in claim 11, wherein said composition is administered for the treatment and/or prevention of a thromboembolic condition selected from the group consisting of arterial restenosis, septicaemia, cancer, and acute inflammation.

13. The method as claimed in claim 11, wherein said composition is administered to a patient undergoing vascular surgery.
